(19) United States
(12) Patent Application Publication
(10) Pub. No.: US 2023/0149685 A1
(43) Pub. Date: May 18, 2023

(54) INTRODUCER WITH SALINE ANTECHAMBER

(71) Applicant: Boston Scientific Scimed, Inc., Maple Grove, MN (US)

(72) Inventors: Troy Anthony Giese, Blaine, MN (US); Christopher J. Koudela, New London, MN (US); Joel T. Eggert, Plymouth, MN (US)

(73) Assignee: Boston Scientific Scimed, Inc., Maple Grove, MN (US)

(21) Appl. No.: 17/988,192

(22) Filed: Nov. 16, 2022

Related U.S. Application Data
(60) Provisional application No. 63/279,791, filed on Nov. 16, 2021.

(51) Int. Cl. A61M 39/02; A61M 39/06

(52) U.S. Cl. CPC A61M 39/0208; A61M 39/0606; A61M 2039/0232; A61M 2039/0235

(57) ABSTRACT

An introducer assembly is adapted for deploying a medical device. The introducer assembly includes an introducer body that is adapted to accommodate a volume of blood therein. An antechamber is coupled with the introducer body. A first hemostasis valve is disposed proximal of the antechamber and fluidly couples the introducer body with the antechamber. A second hemostasis valve is disposed distal of the antechamber and is adapted to allow the medical device to pass therethrough.

### RELATED APPLICATIONS

This application claims the benefit of priority under 35 U.S.C. § 119 of U.S. Provisional Application No. 63/279,791, filed Nov. 16, 2021, the entire disclosure of which is hereby incorporated by reference.

### TECHNICAL FIELD

The present disclosure pertains to medical devices, and methods for manufacturing and using medical devices. More particularly, the disclosure is directed to an introducer that includes a saline antechamber.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. As an example, an introducer assembly is adapted for deploying a medical device. The introducer assembly includes an introducer body that is adapted to accommodate a volume of fluid therein and an antechamber that is coupled with the introducer body. A first hemostasis valve is disposed proximal of the antechamber and fluidly couples the introducer body with the antechamber. A second hemostasis valve is disposed distal of the antechamber and is adapted to allow the medical device to pass therethrough.

Additionally or alternatively, the antechamber may include an annular body having a major dimension and a minor dimension, the major dimension perpendicular with an axis of introduction extending through the introducer assembly.

Additionally or alternatively, the axis of introduction may define a path along which a medical device may travel when passing through the introducer assembly.

Additionally or alternatively, the antechamber may be adapted to hold a volume of saline sufficient to completely submerge the path along which the medical device may travel.

Additionally or alternatively, the volume of saline may be sufficient to permit any air remaining in the medical device to be purged into the antechamber before the medical device passes through the first hemostasis valve.

Additionally or alternatively, the antechamber may be coupled with the introducer body such that the major dimension of the antechamber is substantially vertical when the introducer assembly is in use.

Additionally or alternatively, during use, the major dimension of the antechamber may form an angle with the horizon that is in a range of about 60 degrees to about 90 degrees.

Additionally or alternatively, the introducer assembly may further include one or more luer fittings disposed about a periphery of the antechamber.

Additionally or alternatively, one of the one or more luer fittings may be adapted to be used to introduce saline into the antechamber.

Additionally or alternatively, one of the one or more luer fittings may be adapted to be used to bleed air out of the antechamber.

As another example, an assembly includes an axis of introduction defining a path along which a medical device may be introduced through the assembly. The assembly includes an introducer body and an antechamber that is coupled with the introducer body, the antechamber having an annular body with a major dimension and a minor dimension, the major dimension perpendicular with an axis of introduction extending through the assembly. A first hemostasis valve is disposed between the introducer body and the antechamber, and a second hemostasis valve is disposed distal of the antechamber.

Additionally or alternatively, the antechamber may be adapted to hold a volume of saline sufficient to completely submerge the path along which the medical device may travel.

Additionally or alternatively, the volume of saline may be sufficient to permit any air remaining in the medical device to be purged into the antechamber before the medical device passes through the first hemostasis valve.

Additionally or alternatively, the assembly may further include one or more luer fittings disposed about a periphery of the antechamber.

Additionally or alternatively, one of the one or more luer fittings may be adapted for introducing saline into the antechamber.

Additionally or alternatively, one of the one or more luer fittings may be adapted for bleeding air out of the antechamber.

As another example, an introducer is adapted for deploying a medical device. The introducer includes an introducer body and an antechamber that is coupled with the introducer body, the antechamber adapted to hold a volume of saline sufficient to completely submerge a path along which the medical device may travel when passing through the introducer and to permit any air remaining in the medical device to be purged into the antechamber before the medical device passes through the first hemostasis valve. A first hemostasis valve is disposed proximal of the antechamber and fluidly couples the introducer body with the antechamber. A second hemostasis valve is disposed distal of the antechamber and is adapted to allow the medical device to pass therethrough.

Additionally or alternatively, the antechamber may include an annular body having a major dimension and a minor dimension, the major dimension perpendicular with an axis of introduction extending through the introducer.

Additionally or alternatively, the axis of introduction may define a path along which a medical device may travel when passing through the introducer assembly.

Additionally or alternatively, the antechamber may be coupled with the introducer body such that the major dimension of the antechamber is substantially vertical when the introducer assembly is in use.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of an illustrative introducer assembly;

FIG. 2 is a cross-sectional view taken along line 2-2 of FIG. 1;

FIG. 3 is an exploded perspective view of a portion of the illustrative introducer assembly of FIG. 1; and

FIG. 4 is a partial cross-sectional view of an illustrative introducer assembly.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

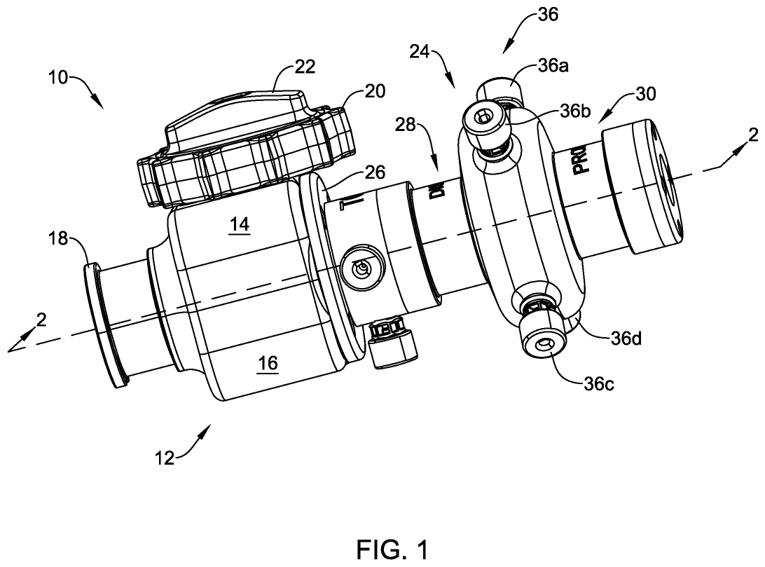

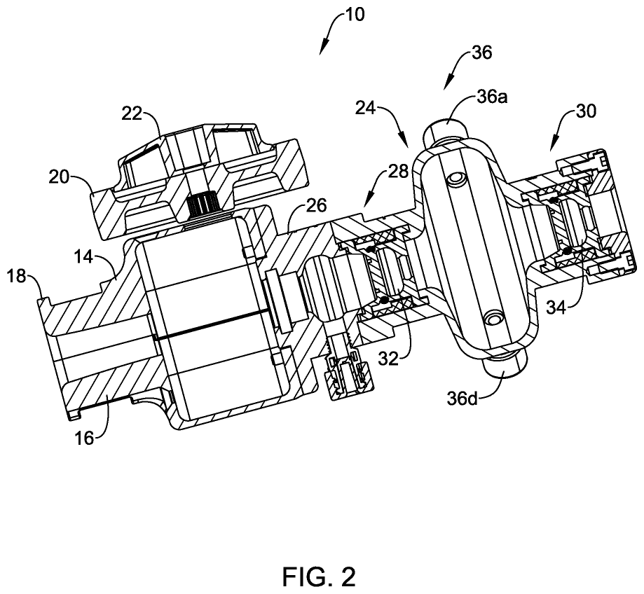

FIG. 1 is a perspective view of an illustrative introducer assembly 10 and FIG. 2 is a cross-sectional view thereof, taken along the 2-2 line of FIG. 1. The illustrative introducer assembly 10 may be used for introducing any of a variety of medical devices into the human body. Examples of suitable medical devices that may be introduced using the introducer assembly 10 include but are not limited to various delivery systems such as delivery systems for delivering mitral valve repair or replacement, tricuspid valve repair or replacement, aortic valve repair or replacement. Delivery systems for other structural heart devices may also be introduced using the introducer assembly 10. Other surgical tool or device such as stent delivery devices, renal denervation devices, angiography devices, cardiac mapping devices and cardiac ablation devices may also be introduced using the introducer assembly 10.

In some instances, as will be discussed, the introducer assembly 10 is adapted to permit a medical device to be passed through the introducer assembly 10 without allowing any air to follow the medical device into the body. The introducer assembly 10 may be adapted to be able to capture any air that enters the introducer assembly 10 as a result of being purged from the medical device itself. For example, a variety of medical devices have exposed distal tips with mechanisms or cavities that are difficult to completely purge of air via traditional flushing methods. Being able to purge these devices of air within the introducer assembly 10 provides a significant advantage.

The introducer assembly 10 includes an introducer body 12. The introducer body 12 may be adapted, for example, to hold a volume of fluid such as blood, saline, or other blood-compatible fluids within the introducer body 12. As shown, the introducer body 12 may include a first introducer body half 14 and a second introducer body half 16, although in some cases the introducer body 12 may be monolithically formed. In some cases, the introducer body 12 may be formed of three or more distinct components that are individually molded and subsequently secured together. The introducer body 12 includes a distal section 18 that is adapted to be secured to other elements such as a stabilizer or a stand. In some cases, the distal section 18 may be adapted to be secured to a telescoping catheter handle.

The introducer body 12 includes a knob 20 and a knob cover 22. In some cases, the knob 20 may be used to steer the distal end of a catheter (not shown).

The introducer assembly 10 includes an antechamber 24 that may be operably coupled to the introducer body 12 via an antechamber flange 26. The antechamber 24 includes a distal cylindrical portion 28 and a proximal cylindrical portion 30. The distal cylindrical portion 28 may be adapted to accommodate a first valve 32 while the proximal cylindrical portion 30 may be adapted to accommodate a second valve 34. The first valve 32 and the second valve 34 may each be adapted to be able to allow an elongate device to pass through the first valve 32 and the second valve 34 while not permitting fluid to pass through either of the first valve 32 or the second valve 34. In some instances, the first valve 32 may be considered as being a first hemostasis valve 32 and the second valve 34 may be considered as being a second hemostasis valve 34. The first hemostasis valve 32 and the second hemostasis valve 34 may cooperate to hold a volume of saline within the antechamber 24.

In some instances, the antechamber 24 includes one or more luer fittings 36, individually labeled as 36a, 36b, 36c, 36d. In some cases, the one or more luer fittings 36 may include removable caps. At least one of the one or more luer fittings 36 may be adapted to allow an operator to connect a source of saline in order to fill the antechamber 24 with saline. At least one of the one or more luer fittings 36 may be adapted to allow an operator to connect a vacuum to one of the luer fittings 36 in order to evacuate air from within an interior of the antechamber 24. This may include evacuating the air originally present within the antechamber 24 before any saline was added to the antechamber 24. In some cases, this may include evacuating air that was introduced subsequent to filling the antechamber 24 with saline, including air that was purged into the saline from a medical device being introduced through the introducer assembly 10.

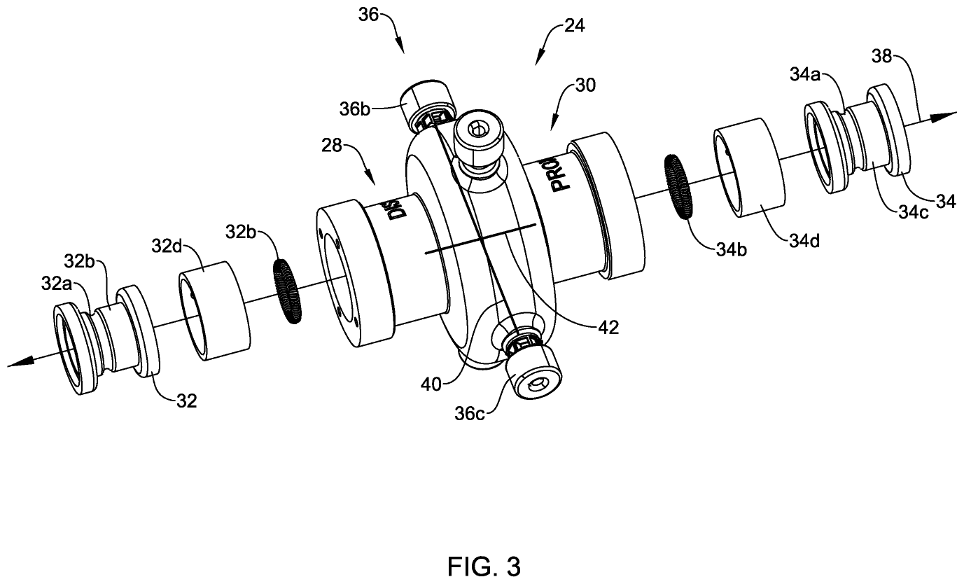

FIG. 3 is an exploded perspective view of a portion of the illustrative introducer assembly 10. FIG. 3 shows the first hemostasis valve 32 and the second hemostasis valve 34 in an exploded fashion relative to the antechamber 24. In some cases, the first hemostasis valve 32 and the second hemostasis valve 34 may include additional components that sealingly secure the first hemostasis valve 32 and the second hemostasis valve 34 in position within the distal cylindrical portion 28 and the proximal cylindrical portion 30, respectively. As shown, the first hemostasis valve 32 includes an annular groove 32a that is adapted to accommodate a spring 32b. The first hemostasis valve 32 includes a recessed portion 32c that is adapted to accommodate an annular spacer 32d. Similarly, the second hemostasis valve 34 includes an annular groove 34a that is adapted to accommodate a spring 34b. The second hemostasis valve 34 includes a recessed portion 34c that is adapted to accommodate an annular spacer 34d. In some cases, the first hemostasis valve 32 and/or the second hemostasis valve 34 may have different outer profiles that do not utilize the springs 32b, 34b or the annular spaces 32d, 34d.

In use, the introducer assembly 10 would be placed in position relative to a patient and coupled with one or more elements that penetrate into the vasculature of the patient. The antechamber 24 would be filled with saline. Any air remaining in the antechamber 24 would be evacuated. This may include air introduced into the antechamber 24 by purging air out of any medical device that will be introduced into the patient through the introducer assembly 10. For example, a medical device having a distal tip that is not easily purged of air may have its distal tip inserted through the second hemostasis valve 34 and into the antechamber 24. Once the distal tip has been purged of air, any air possible may be evacuated from the antechamber 24 by applying a vacuum to one or more of the luer fittings 36. The medical device may then be advanced through the first hemostasis valve 32, through the introducer body 12 and into the patient.

It will be appreciated that the saline present within the antechamber 24, particularly if the antechamber 24 is full or at least substantially full of saline, means that no air will be entrained by any medical device passing through the antechamber 24. This provides a true wet to wet introduction into the blood pool (within the introducer body 12). This means that if there are any leaks through the first hemostasis valve 32, any such leaks would introduce saline, not air, into the blood pool and thus would not be problematic. The antechamber 24 is adapted such that a final purge of the medical device may be made into the saline within the antechamber 24.

As shown in FIG. 3, an axis of introduction 38 extends through the antechamber 24 and hence through the introducer assembly 10. The axis of introduction 38 may be considered not only as a longitudinal axis of the introducer assembly 10, but also as representing a path that a medical device being introduced through the introducer assembly 10 would travel.

The antechamber 24 may be considered as having a toroidal or doughnut-shaped profile including a major dimension 40 and a minor dimension 42 that is orthogonal to the major dimension 40. In some cases, the major dimension 40 may be considered as being orthogonal or at least substantially orthogonal to the axis of introduction 38. Accordingly, the minor dimension 42 may be considered as being parallel or at least substantially parallel with the axis of introduction 38. In this, substantially orthogonal may be considered as being within 10 degrees plus or minus of orthogonal. Substantially parallel may be considered as being within 10 degrees plus or minus of parallel. In some cases, during use, the axis of introduction 38 may be considered as being non-parallel with the horizon, and may be tilted at an angle of 0 to 20 degrees or 0 to 30 degrees with respect to the horizon, with a proximal end of the introducer assembly 10 being higher than a distal end of the introducer assembly 10. As a result, during use, the major dimension 40 of the antechamber 24 may be considered as being substantially vertical, such that the antechamber 24 is substantially filed with fluid. Substantially vertical may be considered as the major dimension 40 of the antechamber 24 forming an angle of 60 degrees to 90 degrees with the horizon, for example.

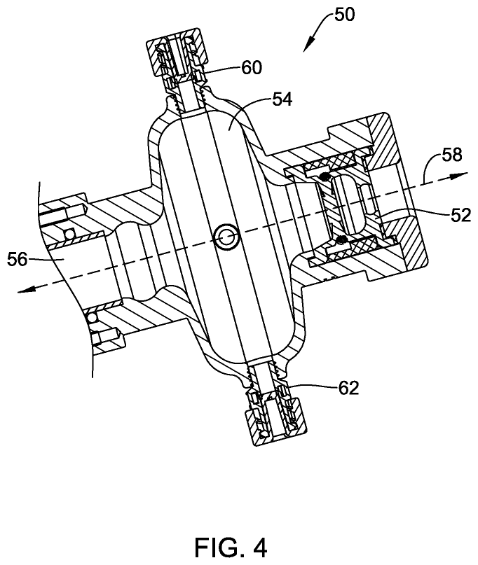

FIG. 4 is a partial cross-sectional view of an illustrative introducer assembly 50 that can provide similar functionality to the introducer assembly 10, but includes only a single hemostasis valve 52. The introducer assembly 50 includes a distal chamber 54 that is disposed between the hemostasis valve 52 and a drip line 56. The distal chamber 54 resembles the antechamber 24, but is adapted to accommodate a sufficient volume of saline such that any air trapped within the distal chamber 54 will rise to the top of the distal chamber 54 and be out of the way of an axis of introduction 58 extending through the introducer assembly 50. The axis of introduction 58 illustrates the path a medical device would take when being advanced through the introducer assembly 50.

The introducer assembly 50 includes at least one luer fitting 60 that may be used to purge air out of the distal chamber 54 and at least one luer fitting 62 that may be used to introduce saline into the distal chamber 54. This may include evacuating the air originally present within the distal chamber 54 before any saline was added to the antechamber 24. In some cases, this may include evacuating air that was introduced subsequent to filling the distal chamber 54 with saline, including air that was purged into the saline from a medical device being introduced through the introducer assembly 50.

The introducer assembly 10 may be formed of any of a variety of different materials, including polymers. In some cases, the introducer assembly 10 or individual components thereof may be formed of a polymeric material such as polycarbonate, acrylic or polyethylene. In some cases, the introducer assembly 10 or individual components thereof may be formed of a metallic material such as stainless steel, for example.

Additional examples of suitable polymers include but are not limited to polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An introducer assembly adapted for deploying a medical device, the introducer assembly comprising:
an introducer body adapted to accommodate a volume of fluid therein;
an antechamber coupled with the introducer body;
a first hemostasis valve disposed proximal of the antechamber and fluidly coupling the introducer body with the antechamber;
a second hemostasis valve disposed distal of the antechamber and adapted to allow the medical device to pass therethrough.

2. The introducer assembly of claim 1, wherein the antechamber comprises an annular body having a major dimension and a minor dimension, the major dimension perpendicular with an axis of introduction extending through the introducer assembly.

3. The introducer assembly of claim 2, wherein the axis of introduction defines a path along which a medical device may travel when passing through the introducer assembly.

4. The introducer assembly of claim 3, wherein the antechamber is adapted to hold a volume of saline sufficient to completely submerge the path along which the medical device may travel.

5. The introducer assembly of claim 4, wherein the volume of saline is sufficient to permit any air remaining in the medical device to be purged into the antechamber before the medical device passes through the first hemostasis valve.

6. The introducer assembly of claim 1, wherein the antechamber is coupled with the introducer body such that the major dimension of the antechamber is substantially vertical when the introducer assembly is in use.

7. The introducer assembly of claim 6, wherein during use, the major dimension of the antechamber forms an angle with the horizon that is in a range of about 60 degrees to about 90 degrees.

8. The introducer assembly of claim 1, further comprising one or more luer fittings disposed about a periphery of the antechamber.

9. The introducer assembly of claim 8, wherein one of the one or more luer fittings is adapted to be used to introduce saline into the antechamber.

10. The introducer assembly of claim 8, wherein one of the one or more luer fittings is adapted to be used to bleed air out of the antechamber.

11. An assembly including an axis of introduction defining a path along which a medical device may be introduced through the assembly, the assembly comprising:
an introducer body;
an antechamber coupled with the introducer body, the antechamber having an annular body with a major dimension and a minor dimension, the major dimension perpendicular with an axis of introduction extending through the assembly;
a first hemostasis valve disposed between the introducer body and the antechamber;
a second hemostasis valve disposed distal of the antechamber.

12. The assembly of claim 11, wherein the antechamber is adapted to hold a volume of saline sufficient to completely submerge the path along with the medical device may travel.

13. The assembly of claim 12, wherein the volume of saline is sufficient to permit any air remaining in the medical device to be purged into the antechamber before the medical device passes through the first hemostasis valve.

14. The assembly of claim 11, further comprising one or more luer fittings disposed about a periphery of the antechamber.

15. The assembly of claim 14, wherein one of the one or more luer fittings is adapted for introducing saline into the antechamber.

16. The assembly of claim 14, wherein one of the one or more luer fittings is adapted for bleeding air out of the antechamber.

17. An introducer adapted for deploying a medical device, the introducer comprising:
an introducer body;
an antechamber coupled with the introducer body, the antechamber adapted to hold a volume of saline sufficient to completely submerge a path along which the medical device may travel when passing through the introducer and to permit any air remaining in the medical device to be purged into the antechamber before the medical device passes through the first hemostasis valve;
a first hemostasis valve disposed proximal of the antechamber and fluidly coupling the introducer body with the antechamber;
a second hemostasis valve disposed distal of the antechamber and adapted to allow the medical device to pass therethrough.

18. The introducer of claim 17, wherein the antechamber comprises an annular body having a major dimension and a minor dimension, the major dimension perpendicular with an axis of introduction extending through the introducer.

19. The introducer of claim 18, wherein the axis of introduction defines a path along which a medical device may travel when passing through the introducer assembly.

20. The introducer of claim 17, wherein the antechamber is coupled with the introducer body such that the major dimension of the antechamber is substantially vertical when the introducer assembly is in use.
